(19) United States
(12) Patent Application Publication
(10) Pub. No.: US 2013/0211472 A1
(43) Pub. Date: Aug. 15, 2013

(54) CARDIAC RESYNCHRONIZATION THERAPY OPTIMIZATION

(71) Applicant: Andreas Blomqvist, Taby (SE)

(72) Inventor: Andreas Blomqvist, Taby (SE)

(73) Assignee: ST. JUDE MEDICAL AB, Jarfalla (SE)

(21) Appl. No.: 13/880,345

(22) PCT Filed: Oct. 27, 2010
(86) PCT No.: PCT/SE2010/051165

§ 371 Date: Apr. 18, 2013

(51) Int. Cl. A61N 1/365

(52) U.S. Cl. CPC A61N 1/36585
USPC 607/18; 607/17

(57) ABSTRACT

An implantable medical device, IMD, (100) conducts CRT settings searches at multiple CRT settings search periods during an optimization time period by testing different candidate CRT settings and selecting the optimal CRT setting based on output signals of a hemodynamic sensor (240). The respective optimal CRT settings determined during the optimization time period are employed in order to predict at least one future optimal CRT setting that can be used by the IMD (100) following the end of the optimization time period. The IMD (100) then generates and applies pacing pulses to a subject's (5) heart (10) according to a CRT setting of the at least one future optimal CRT setting. The embodiments therefore enable efficient cardiac resynchronization therapy without any sensor readings after the end of the optimization time period and can therefore provide cardiac resynchronization therapy even if the hemodynamic sensor (240) becomes inoperable.

### TECHNICAL FIELD

The present invention generally relates to cardiac resynchronization therapy (CRT) optimization, and in particular to an implantable medical device operating according to predicted, optimized CRT settings.

### BACKGROUND

It is estimated that nearly 5 million Americans have heart failure with 400.000 new cases every year. The prevalence of heart failure approximately doubles with each decade of life. One of the most important means of treating heart failure is cardiac resynchronization therapy (CRT). Although CRT is a very effective way of treating heart failure in most patients there is a large percentage for which the CRT has no apparent effect at all. Different estimates of the size of this group of so called “non-responders” exist, but it is generally believed to be in the vicinity of 25% of all patients equipped with a CRT, but there are numbers reported to be as high as 33%.

Implantable medical devices (IMDs), such as pacemakers, are today used for patients suffering from various cardiac disorders and malfunctions. In the field of IMDs, device-based optimization of CRT settings, including atrioventricular delay (AVD) and interventricular delay (VVD), is likely to be one of the most potent tools in fighting non-responders and also in improving CRT efficacy.

Many sensor-based techniques have been suggested over the years to be used in order to find optimal AVD and VVD, but the “gold standard” to many physicians still is left ventricular

$${( \frac{d P}{d t} )}_{ma x} .$$

A problem, though, is that it is hard to manufacture sensors capable of delivering left ventricular

$${( \frac{d P}{d t} )}_{ma x}$$

in chronic settings as the sensors generally become inoperable or unpredictable after some time in the human body. In addition, most CRT optimization algorithms of today involve the constant scanning of AVDs and VVDs causing a significant time in a hemodynamic sub-optimal setting for the patient during each search period.

U.S. Pat. No. 7,027,866 discloses an IMD having a pressure sensor in the right ventricle. Continuous or periodic measurements of

$$( \frac{d P}{d t} )$$

values are conducted for various AVDs/VVDs to be tested. A look-up table is generated that stores the AVD/VVD results, the

$$( \frac{d P}{d t} )$$

values and the heart rates during the measurements. This look-up table can be used by the IMD if the pressure sensor later on becomes inoperable or is removed. In such a case, the current heart rate is used to select which AVD/VVD to use in order to achieve optimal hemodynamic response for the given heart rate.

U.S. Pat. No. 7,184,835 discloses selection of optimal AVD for a patient based on electrical or mechanical events having a predictable relationship with an optimal AVD. Different time parameters are determined for a patient, including time between P-wave and beginning of QRS complex, time between P-wave and onset of pressure increase in the left ventricular contraction and time between P-wave and R-wave of the QRS complex. Different AVDs are tested for the patient and

$$( \frac{d P}{d t} )$$

is determined for each AVD in order to find optimal AVD. The optimal AVD is then associated with the determined time parameters. This procedure is conducted for multiple patients to obtain a look-up table that lists optimal AVDs for the different time parameters.

US 2007/0129764 relates to a pacemaker capable of optimizing AVD based on

$$( \frac{d P}{d t} )$$

from a physiologic sensor. An optimization procedure is conducted by testing, given a particular heart rate, various pacing intervals and recording the sensor output for each pacing interval. This procedure is typically conducted for at least one other heart rate. In such a case, optimal AVD can be extrapolated or interpolated for other heart rates based on the tested heart rates. The results, including extrapolated or interpolated data, are stored and used by the pacemaker for adjustment of AVD.

### SUMMARY

There is still a need in the art to provide an efficient CRT optimization and in particular such CRT optimization that can provide reliable CRT settings even when hemondynamic sensor readings are no longer available for the IMD.

It is a general objective to provide efficient cardiac resynchronization therapy.

This and other objectives are met by embodiments as disclosed herein.

Briefly, an aspect relates to a system for determining CRT settings for an IMD having a lead and sensor connector connectable to at least a first cardiac lead and a second cardiac lead. The first cardiac lead is implantable in or in connection with a first heart chamber of a heart in a subject and has at least one pacing and sensing electrode. The second cardiac lead correspondingly comprises at least one pacing and sensing electrode and is implantable in or in connection with a second heart chamber of the heart. The lead and sensor connector is further connectable to at least one hemodynamic sensor configured to generate output signals representative of the hemodynamic status of the subject.

The IMD also comprises a pacing pulse generator configured to generate pacing pulses applicable to the first and second heart chambers by means of the first and second cardiac leads. A controller is connected to the pacing pulse generator and configured to generate control signals to control the pacing pulse generator to generate pacing pulses according to multiple different candidate CRT settings of a programmable CRT parameter. The multiple CRT settings are tested at multiple CRT settings search periods during an optimization time period.

A settings optimizer of the system is configured to determine a respective optimal CRT setting for a defined heart rate range of the heart and for each of the multiple CRT settings search periods. The settings optimizer determines the optimal CRT setting based on the multiple different candidate CRT settings and based on the output signals from the hemodynaic sensor and therefore based on the hemodynamic status of the subject.

The system also comprises a settings predictor configured to predict at least one future optimal CRT setting for the defined heart rate range. The settings predictor conducts this settings prediction based on the respective optimal CRT settings determined by the settings optimizer. The at least one future optimal CRT setting is stored in a memory of the IMD and is applicable following the end of the optimization time period. Thus, the controller of the IMD then generates, following the end of the optimization time period, control signals to control the pacing pulse generator to generate pacing pulses according to a CRT setting selected among the at least one future optimal CRT setting.

Optimal CRT setting can therefore be predicted and calculated based on previously determined optimal CRT settings. This means that no sensor output signals are needed in order to obtain an optimal CRT setting following the end of the optimization time period. This means that the system enables efficient cardiac resynchronization therapy even if the hemodynamic sensor becomes inoperable or unpredictable some time after implantation.

Another aspect relates to a method for determining CRT settings for an IMD. The method involves generating and applying pacing pulses to two heart chambers of a subject's heart by the IMD according a candidate CRT setting of a programmable CRT parameter for the IMD. A hemodynamic sensor generates an output signal representative of the hemodynamic status of the subject due to pacing according to the candidate CRT setting. This procedure is repeated for multiple different candidate CRT settings at a CRT settings search period during an optimization time period. An optimal CRT setting is determined for the CRT setting search period based on the multiple different candidate CRT settings and also based on the output signals from the hemodynamic sensor. The procedure is repeated for multiple CRT settings search periods during the optimization time period to thereby obtain multiple optimal CRT settings for a defined heart rate range. These multiple optimal CRT settings are employed to predict at least one future optimal CRT setting for the defined heart rate range. The at least one future optimal CRT setting is stored in an IMD memory and can be used by the IMD to generate and apply pacing pulses to the two heart chambers following the end of the optimization time period.

### BRIEF DESCRIPTION OF THE DRAWINGS

The invention, together with further objects and advantages thereof, may best be understood by making reference to the following description taken together with the accompanying drawings, in which:

FIG. 1 is an overview of a system for determining CRT settings according to an embodiment illustrated at least partly implanted in a human subject;

FIG. 2 is a schematic block diagram of an embodiment of an implantable medical device to be used in a system for determining CRT settings;

FIG. 3 is a schematic block diagram of another embodiment of an implantable medical device to be used in a system for determining CRT settings;

FIG. 4 is a schematic block diagram of an embodiment of a programmer to be used together with the implantable medical device of FIG. 3 in a system for determining CRT settings;

FIG. 5 is an illustration of a human heart with implantable cardiac leads and an implantable hemodynamic sensor according to an embodiment;

FIG. 6 schematically illustrates the concept of determining optimal CRT settings for different heart rates according to an embodiment;

FIG. 7 is a diagram illustrating predicting future optimal CRT settings according to an embodiment;

FIG. 8 is a diagram illustrating the changes in optimal AVD over time for 40 CRT patients;

FIG. 9 is a diagram illustrating the changes in optimal VVD over time for 40 CRT patients;

FIG. 10 is a flow diagram illustrating a method of determining CRT settings for an implantable medical device;

FIG. 11 is a flow diagram illustrating an embodiment of the predicting step in the method of FIG. 10; and

FIG. 12 is a flow diagram illustrating an additional step of the method in FIG. 10.

### DETAILED DESCRIPTION

Throughout the drawings, the same reference numbers are used for similar or corresponding elements.

The embodiments generally relate to implantable medical devices and in particular to such implantable medical devices having adjustable cardiac resynchronization therapy (CRT) settings. In particular, the embodiments disclose the determination of such CRT settings for an implantable medical device even when no hemodynamic status feedback is available for the implantable medical device. Thus, the embodiments enable a prediction of future optimal CRT settings for the implantable medical device based on previously verified optimal CRT settings. Hence, the embodiments can significantly improve the patient specificity in terms of defining suitable CRT settings for the implantable medical device, thereby reducing the non-responder rate among CRT patients.

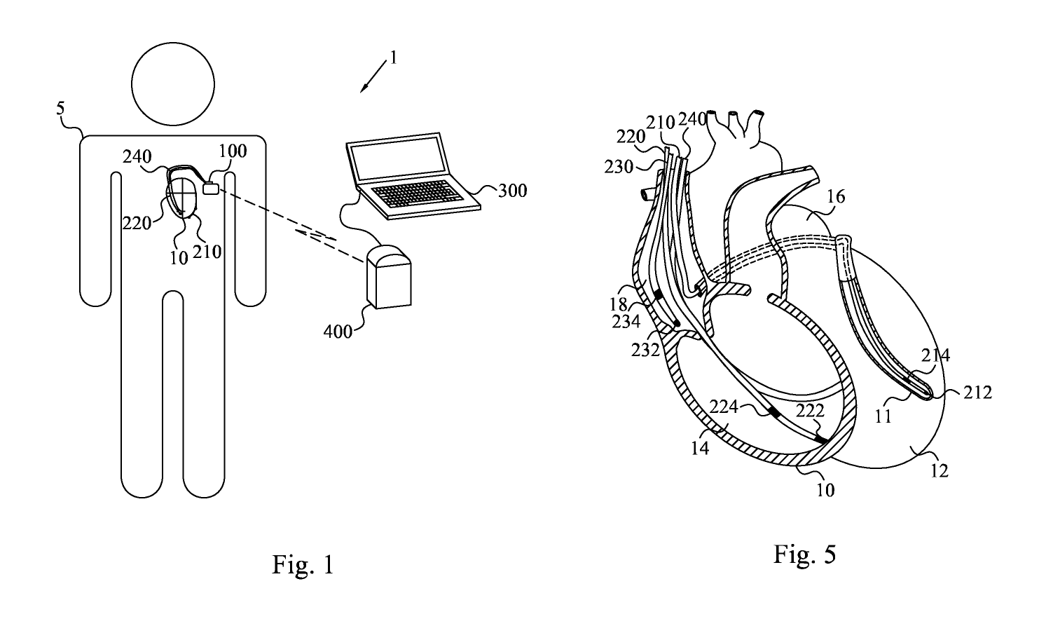

FIG. 1 is a schematic overview of a system 1 for determining CRT settings for an implantable medical device (IMD) 100. In the figure, the IMD 100 is illustrated implanted in a human patient 5 and as a device that monitors and/or provides therapy to the heart 10 of the patient 5. The patient 5 must not necessarily be a human patient but can instead be an animal patient, in particular a mammalian patient, in which an IMD 100 can be implanted. The IMD 100 can be in the form of a pacemaker, a cardiac defibrillator or a cardioverter, such as an implantable cardioverter-defibrillator (ICD), as long as the IMD 100 is capable of providing cardiac resynchronization therapy to the patient 5. The IMD 100 is, in operation, connected to multiple, i.e. at least two cardiac leads 210, 220 inserted into or in connection with different heart chambers, the right ventricle and left ventricle in the figure. The IMD 100 is also connectable to a hemodynamic sensor 240, exemplified by a pressure wire 240 in the figure.

The figure also illustrates an external data processing device 300, such as programmer or clinician's workstation, that can communicate with the IMD 100, optionally through a communication device 400 that operates similar to a base station on behalf of the data processing device 300. As is well known in the art, such a data processing device 300 can be employed for transmitting IMD programming commands causing a reprogramming of different operation parameters and modes of the IMD 100. Furthermore, the IMD 100 can upload diagnostic data descriptive of different medical parameters or device operation parameters collected by the IMD 100. Such uploaded data may optionally be further processed in the data processing device 300 before display to a clinician. In the light of the present invention, the IMD 100 can transmit diagnostic data in terms of values representative of hemodynamic status and information of candidate CRT parameter values. The data processing device 300 can further be used to transmit future optimal CRT settings to the IMD 100 to use when setting a CRT parameter of the IMD 100. In an embodiment, the system 1 comprises the IMD 100 but not the data processing device 300, whereas in another, distributed embodiment a part of the system 1 is arranged in the IMD 100 and another part of the system 1 is implemented in the data processing device 300, which is further discussed herein.

According to the embodiments, the settings of at least one programmable CRT parameter of the IMD is determined. The CRT parameter can be any programmable CRT parameter known in the art of IMDs capable of delivering cardiac resynchronization therapy. In the following, the embodiments will be described in more detail in connection with particular examples of such programmable CRT parameters, namely atrioventricular delay (AVD) also denoted atrioventricular interval (AVI) in the art and interventricular delay (VVD) also denoted interventricular interval (VVI). These CRT parameters should, however, merely be seen as illustrative but non-limiting examples of preferred CRT parameters.

The embodiments can be employed to determine the settings of a single programmable CRT parameter of an IMD, such as AVD or VVD. In an alternative approach, the embodiments can determine the settings of multiple programmable CRT parameters of an IMD, such as both AVD and VVD. In the following, reference to the determination of the settings of a programmable CRT parameter also encompasses the determination of the settings of multiple different such CRT parameters.

According to the embodiments optimal CRT setting is determined for a programmable CRT parameter. The expression “optimal” should be interpreted according to the embodiments to relate to achieving an improved effect to the subject as compared to a less optimal CRT setting. The optimization thereby utilizes an optimization parameter representative of hemodynamic status of the subject in order to try to find the CRT setting that leads to “better” or “optimal” hemodynamic status among the tested CRT settings. Optimal CRT setting should therefore be interpreted herein to a CRT setting that leads to an improved hemodynamic status as compared other tested CRT settings that give less favorable hemodynamic status for the subject.

In a general aspect, the system for determining CRT settings for an IMD comprises the IMD having a lead and sensor connector that is connectable to at least a first cardiac lead implantable in or in connection with a first heart chamber of the subject's heart. This first cardiac lead comprises at least one pacing and sensing electrode. The lead and sensor connector is also connectable to a second cardiac lead implantable in or in connection with a second heart chamber of the heart and has at least one pacing and sensing electrode. Furthermore, the lead and sensor connector is connectable to a hemodynamic sensor configured to generate output signals representative of the hemodynamic status of the subject. The IMD also comprises at least one pulse generator connected to the lead and sensor connector and configured to generate pacing pulses applicable to the first heart chamber and the second heart chamber by means of the first cardiac lead and the second cardiac lead, respectively. A controller of the IMD is connected to the at least one pacing pulse generator and is configured to generate control signals. The at least one pacing pulse generator is responsive to these control signals and generates, based on the control signals and at multiple CRT settings search periods during an optimization time period, pacing pulses according to multiple different candidate CRT settings of a programmable CRT parameter.

The system also comprises a settings optimizer configured to determine a respective optimal CRT setting for a defined heart rate range of the heart and for each CRT settings search period of the multiple CRT settings search periods. The determination of the respective optimal CRT settings is furthermore performed based on the multiple different candidate CRT settings and based on the output signals from the hemodynamic sensor.

A settings predictor of the system is configured to predict at least one future optimal CRT setting for the defined heart rate and based on the respective optimal CRT settings determined by the settings optimizer. This at least one future optimal CRT setting predicted by the settings predictor is stored in a memory of the IMD and can be used by the controller to generate control signals following the end of the optimization time period. These control signals will control the at least one pacing pulse generator to generate pacing pulses according to a CRT setting selected among the at least one future optimal CRT setting stored in the IMD memory.

Thus, in the general aspect the IMD tests various candidate CRT settings of the programmable CRT parameter at multiple CRT settings search periods during the optimization time period. The optimal CRT setting at each of the multiple CRT settings search periods is determined or selected based on the hemodynamic status of the subject so that the CRT setting of the various candidate CRT settings resulting in best hemodynamic status will be the optimal CRT setting for that CRT settings search period. As a result, multiple optimal CRT settings each determined at a respective CRT settings search period during the optimization time period are obtained. These multiple optimal CRT settings are then processed to predict future optimal CRT settings that can be used when the optimization time period has ended. In such a case, there is no need to search for optimal CRT settings based on any hemodynamic status monitoring by the hemodynamic sensor. In clear contrast, one of the calculated future optimal CRT settings can be used directly for programming the CRT parameter in the IMD.

Embodiments of the general aspect will now be further described in connection with the drawings.

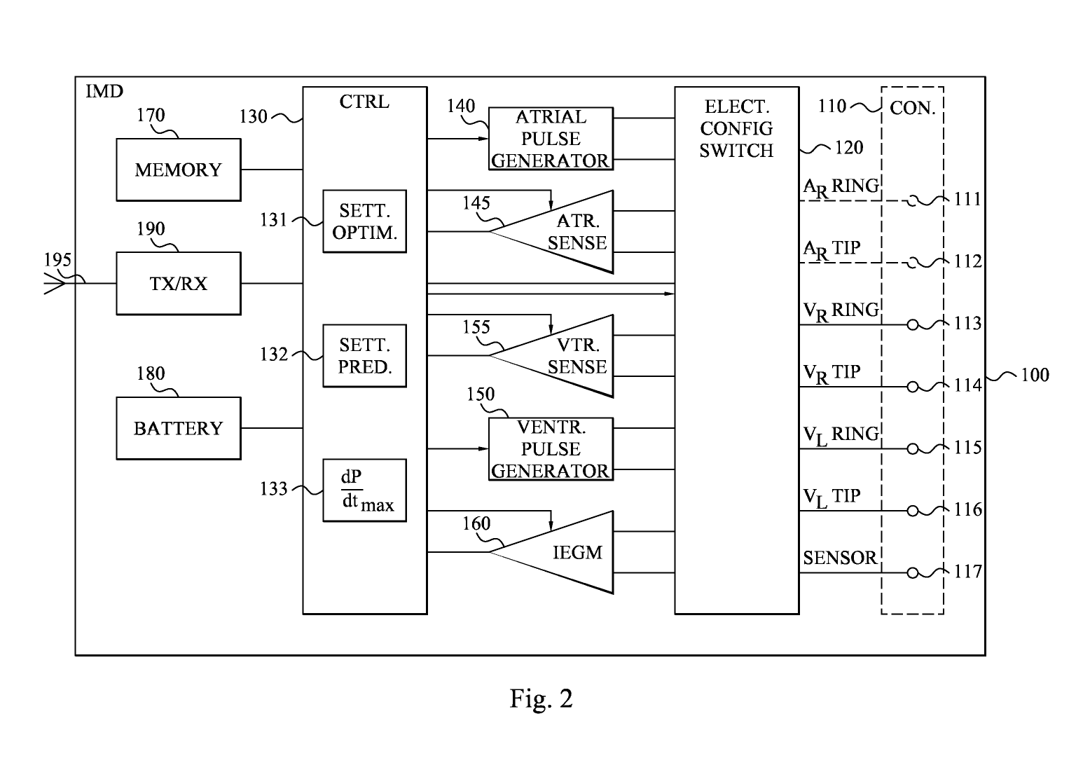

FIG. 2 illustrates an embodiment of an IMD 100 suitable for delivering cardiac resynchronization therapy to a heart of a subject. The figure is a simplified block diagram depicting various components of the IMD 100. While a particular multi-chamber device is shown in the figure, it is to be appreciated and understood that this is done merely for illustrative purposes. Thus, the techniques and methods described below can be implemented in connection with other suitably configured IMDs. Accordingly, the person skilled in the art can readily duplicate, eliminate, or disable the appropriate circuitry in any desired combination to provide an IMD capable of treating the appropriate heart chamber(s) with cardiac resynchronization therapy.

The IMD 100 comprises a housing, often denoted as can or case in the art. The housing can act as return electrode for unipolar leads, which is well known in the art. The IMD 100 also comprises a lead and sensor connector or input/output (I/O) 110 having, in this embodiment, a plurality of terminals 111-117. The terminals 111-116 are configured to be connected to matching electrode terminals of cardiac leads connectable to the IMD 100 and the lead and sensor connector 110. Optionally, the lead and sensor connector 110 comprises at least one dedicated terminal 117 arranged to be connected to a matching terminal of a hemodynamic sensor. This at least one terminal 117 therefore receives the output signals of the hemodynamic sensor representative of the hemodynamic status of the subject.

With reference to FIGS. 2 and 5, the lead and sensor connector 110 is configured to be, during operation in the subject body, electrically connectable to, in this particular example, a right atrial lead 230, a right ventricular lead 220 and a left ventricular lead 210. The lead and sensor connector 110 consequently comprises terminals 111, 112 that are electrically connected to matching electrode terminals of the atrial lead 230 when the atrial lead 230 is introduced in the lead and sensor connector 110. For instance, one of these terminals 112 can be designed to be connected to a right atrial tip terminal of the atrial lead 230, which in turn is electrically connected through a conductor running along the lead body to a tip electrode 232 present at the distal end of the atrial lead 230 in the right atrium 18 of the heart 10. A corresponding terminal 111 is then connected to a right atrial ring terminal of the atrial lead 230 that is electrically connected by another conductor in the lead body to a ring electrode 234 present in connection with the distal part of the atrial lead 230, though generally distanced somewhat towards the proximal lead end as compared to the tip electrode 232.

In an alternative implementation, the IMD 100 is not connectable to a right atrial lead 230 but instead to a left atrial lead configured for implantation in the left atrium 16. A further possibility is to have an IMD 100 with a lead and sensor connector 110 having sufficient terminals to allow the IMD 100 to be electrically connectable to both a right atrial lead 230 and a left atrial lead. Though, it is generally preferred to have at least one electrically connectable atrial lead in order to enable atrial sensing and pacing, the IMD 100 does not necessarily have to be connectable to any atrial leads. In such a case, the terminals 111, 112 of the lead and sensor connector 110 can be omitted.

In order to support left chamber sensing and pacing, the lead and sensor connector 110 further comprises a left ventricular tip terminal 116 and a left ventricular ring terminal 115, which are adapted for electric connection to a left ventricular tip electrode 212 and a left ventricular ring electrode 214 of the left ventricular lead 210 implantable in or in connection with the left ventricle 12, see FIG. 5. A left ventricular lead 210 is typically implanted in the coronary venous system 11 for safety reasons although implantation inside the left ventricle 12 has been proposed in the art. In the following, “left ventricular lead” 210 is used to describe a cardiac lead designed to provide sensing and pacing functions to the left ventricle 12 regardless of its particular implantation site, i.e. inside the left ventricle 12 or in the coronary venous system 11.

Right chamber sensing and pacing can be achieved if the lead and sensor connector 110 comprises a right ventricular tip terminal 114 and a right ventricular ring terminal 113, which are adapted for electric connection to a right ventricular tip electrode 222 and a right ventricular ring electrode 224 of the right ventricular lead 220 implantable in the right ventricle 14.

The IMD 100 and the lead and sensor connector 110 can be designed to provide cardiac resynchronization therapy in terms of achieving ventricular sensing and pacing and thereby adjust the VVD. In such a case, the lead and sensor connector 110 comprises the terminals 113-116 and does not necessarily need to be connected to any atrial lead 230. The terminals 111, 112 can therefore be omitted.

If the IMD 100 instead is designed to provide cardiac resynchronization therapy in terms of achieving atrial and ventricular sensing and pacing and adjust the AVD, it is sufficient that the lead and sensor connector 110 is connectable to a single ventricular lead. In such a case, the left ventricular lead 210 and the terminals 115, 116 designed to be connected to the left ventricular lead 210 can be omitted or the right ventricular lead 220 and its matching terminals 113, 114 can be omitted.

IMDs 100 capable of adjusting both AVD and VVD are preferably connected to both an atrial lead 230, a right ventricular lead 220 and a left ventricular lead 210 as illustrated in FIG. 5.

In FIG. 5 the cardiac leads 210, 220, 230 have been illustrated as bipolar leads, i.e. having a respective tip electrode 212, 222, 232 and a respective ring electrode 214, 224, 234. In an alternative embodiment at least one of the cardiac leads 210, 220, 230 can be a so-called multipolar lead, i.e. having at least three pacing and sensing electrodes, such as a quadropolar lead having a tip electrode three spatially separated ring electrodes.

The lead and sensor connector 110 is further connected to at least one hemodynamic sensor 240. This hemodynamic sensor 240 can be a separate sensor consisting of a sensor lead, wire or catheter connectable to the lead and sensor connector 110. Alternatively, the hemodynamic sensor 240 is arranged onto one of the cardiac leads 210, 220, 230 that comprise the pacing and sensing electrodes 212, 214, 222, 224, 232, 234. There is then no need for any separate sensor lead connectable to the lead and sensor connector 110. In these embodiments, the lead and sensor connector 110 preferably comprises at least one terminal 117 arranged for receiving the output signals from the hemondynamic sensor 240 on the separate sensor lead or on the cardiac lead. In an alternative embodiment, the output signals representative of the hemodynamic status of the subject originates from pacing and sensing electrodes on at least one of the cardiac leads. In such a case, such a pacing and sensing electrode both provides pacing functionality and sensor functionality as is further described herein. There is then generally no need for any dedicated terminal 117 to be connected to the hemodynamic sensor in the lead and sensor connector 110. Instead the terminal out of terminal 111-116 to which the pacing and sensing electrode is connectable also receives the output signal.

If the IMD 100 is connectable to an atrial lead 230, the IMD 100 comprises an atrial pulse generator 140 generating pacing pulses for delivery by the atrial lead(s) preferably through an electrode configuration switch 120. The IMD 100 also comprises a ventricular pulse generator 150 that generates pacing pulses for delivery by the ventricular lead(s) to the left and/or right ventricle.

It is understood that in order to provide stimulation therapy in different heart chambers, the atrial and ventricular pulse generators 140, 150 may include dedicated, independent pulse generators, multiplexed pulse generators, or shared pulse generators. The pulse generators 140, 150 are controlled by a controller 130 via appropriate control signals, respectively, to trigger or inhibit the stimulating pulses.

The controller 130 of the IMD 100 is preferably in the form of a programmable microcontroller 130 that controls the operation of the IMD 100 and in particular the atrial and ventricular pulse generators 140, 150. The controller 130 typically includes a microprocessor, or equivalent control circuitry, designed specifically for controlling the delivery of pacing therapy, and may further include RAM or ROM memory, logic and timing circuitry, state machine circuitry, and I/O circuitry. Typically, the controller 130 is configured to process or monitor input signal as controlled by a program code stored in a designated memory block. The type of controller 130 is not critical to the described implementations. In clear contrast, any suitable controller may be used that carries out the functions described herein. The use of microprocessor-based control circuits for performing timing and data analysis functions are well known in the art.

According to the embodiments, the controller 130 is connected to the atrial and ventricular pulse generators 140, 150 and is configured to generate control signals to which the atrial and ventricular pulse generators 140, 150 respond. In particular, the controller 130 controls the atrial and/or ventricular pulse generator 140, 150 to generate pacing pulses at multiple CRT settings search periods during an optimization time period and furthermore to generate the pacing pulses according to multiple different candidate CRT settings of at least one programmable CRT parameter.

The optimization time period is the time period during which the IMD 100 tests various candidate CRT settings of the at least one programmable CRT parameter, such as AVD and/or VVD. The optimization time period can be a preconfigured time period having a defined length, such as the first X weeks or months following implantation of the IMD 100 in the subject. In such a case, this parameter X can be preconfigured in the IMD 100 at implantation or can be set by the physician during or following implantation. The length of the optimization time period is then preferably set to be sufficient long to determine a sufficient number of optimal CRT settings that enables prediction of future optimal CRT settings based on these determined optimal CRT settings. In general, an optimization time period of at least one month up to several months, such as one year is typically sufficient and more preferably from one month up to six months, such as around three months.

In an alternative or complementary embodiment, the optimization time period is defined based on the operational time of the hemodynamic sensor used in order to determine optimal CRT settings. Generally and depending on the sensor type, a hemodynamic sensor can have limited operational time inside a subject body. This may, for instance, be due to the ingrowth of connective tissue around the sensor that prevents a correct sensor measurement and/or due to that the sensor is based on some catalytic reaction and where the substrate of the catalytic reaction is depleted from an internal store in the hemodynamic sensor. In such a case, the optimization time period can run as long as the hemodynamic sensor is operational and gives correct sensor reading. The length of this operational time can either be defined by the manufacturer of the hemodynamic sensor or is determined by the controller 130 based on previous sensor readings from the hemodynamic sensor. Thus, if the hemodynamic sensor suddenly gives output signals representing a significant change in hemodynamic status and continues to give output signals representing such a significant change for a defined time period and without any accompanying significant change in the electric signals sensed by the pacing and sensing electrodes, the controller 130 can conclude that the hemodynamic sensor is no longer operating correctly.

The controller 130 is configured to conduct searches for optimal CRT setting at multiple different CRT settings search periods during the optimization time period. In such a case, the controller 130 is preferably configured to periodically initiate such a CRT setting search at regular intervals. For instance, the controller 130 can be configured to conduct a CRT setting search once a week during the optimization time period. It is also possible to use non-regular intervals between the CRT setting searches. For instance, optimal CRT settings might change quite rapidly during the first few weeks following implantation and then slowly stabilizes. In such a case, it might be beneficial to perform the CRT setting searches more frequently during the beginning of the optimization time period and less frequently towards the end of the optimization time period. In either way, the controller 130 is preferably configured by the physician or the IMD manufacturer to know when it is time to start a new CRT settings search period and conduct a CRT setting search.

In an alternative embodiment, the start of a CRT setting search is triggered by the reception of a trigger message originating from the external data processing device, see FIG. 1. The physician can then select when the IMD 100 is to conduct a CRT setting search by generating and transmitting, at the data processing device, the trigger message to a receiver 190 of the IMD 100. The controller 130 then triggers a CRT setting search based on the reception of such a trigger message.

The CRT setting search is conducted by the controller 130 generating control signals to cause the atrial pulse generator 140 and/or the ventricular pulse generator 150 to apply pacing pulses to the heart according to a candidate CRT setting, for instance according to a candidate AVD and/or VVD. Any change in the hemodynamic status of the subject due to pacing according to the candidate CRT setting is registered by the hemodynamic sensor and forwarded to the controller 130 through the terminal 117 and the optional switch 120. The hemodynamic sensor could then be continuously operating to produce output signals according to its sampling frequency. This, however, might drain quite a bit of power from the battery 180 of the IMD 100 unless the hemodynamic sensor does not require any power source. In another embodiment, the controller 130 could instead control the hemodynamic sensor to only be active and produce output signals during the CRT setting search periods and in response to the controller 130 outputting a control signal to the atrial pulse generator 140 and/or the ventricular pulse generator 150. The hemodynamic sensor then only produces output signals when they are needed i.e. after and in response to a change in the setting of the programmable CRT parameter.

Once the hemodynamic sensor has output a signal representative of the new hemodynamic status of the subject following pacing according to the candidate CRT setting, the controller 130 controls the atrial pulse generator 140 and/or the ventricular pulse generator 150 to generate pacing pulses according to another candidate CRT setting of the programmable CRT parameter. The new hemodynamic status of the subject is then once more monitored by the hemodynamic sensor.

Thus, at each CRT setting search period multiple different candidate CRT settings are tested and the hemodynamic statuses of the subject obtained following pacing according to the candidate CRT settings are recorded by the hemodynamic sensor.

The number of candidate CRT settings tested could be preconfigured in the IMD 100 or be determined and programmed into the IMD 100 by the physician. For instance, statistics regarding average CRT settings among CRT subjects can be used as a starting point of a given candidate CRT setting. Additional candidate CRT settings can then be selected around this starting point. In a more sophisticated embodiment, the starting point of a given candidate CRT setting could be the optimal CRT setting determined for the previous CRT settings search period when a CRT settings search was last conducted by the IMD 100. In such a case, remaining candidate CRT settings can be selected around this starting point. Furthermore, if statistics regarding the change in a CRT parameter following implantation define that the CRT parameter value typically increases or decreases following implantation, such statistics can be further utilized in order to limit the CRT setting search to candidate CRT settings that are only larger or smaller than the starting point.

The length of the CRT settings search period depends on the number of different candidate CRT settings that should be tested. Thus, the CRT settings search period can be from one or few minutes up to several days or hours. The atrial pulse generator 140 and/or ventricular pulse generator 150 should also continue to generate pacing pulses according to a candidate CRT setting for a sufficient long time in order to have an impact on the hemodynamic status of the subject. Thus, if pacing according to the candidate CRT setting is merely conducted during a few seconds the pacing and the candidate CRT setting will typically have no impact on the subject's hemodynamic status. It is therefore generally preferred to continue pacing according to a candidate CRT setting for at least one or a few minutes in order to get a detectable effect on the hemodynamic status that can be registered by the hemodynamic sensor.

A settings optimizer 131 implemented, in this embodiment, in the IMD 100 receives information of the different tested candidate CRT settings from the controller 130, unless these are predefined and available to the settings optimizer 131. The optimizer 131 also gets access to the respective output signals from the hemodynamic sensor or information relating to hemodynamic status of the subject for the different candidate CRT settings and determined by the controller 130 based on the output signals. Table 1 below illustrates an example of the type of information that the settings optimizer 131 can receive for a CRT settings search period, where the output signal from the hemodynamic sensor are in this case representative of left ventricular (LV) dP/dtₘₐₓ.

TABLE 1
input to settings optimizer

| AVD (ms) | LV dP/dtₘₐₓ (mmHg/s) |
|---|---|
| 50 | 1435 |
| 75 | 1579 |
| 100 | 1602 |
| 125 | 1512 |
| 150 | 1399 |

The settings optimizer 131 then investigates the output signals from the hemodynamic sensor or the information representative thereof in order to determine the optimal CRT setting that will result in the best hemodynamic status of the subject as assessed based on the output signals from the hemodynamic sensor. In an embodiment, the settings optimizer 131 determines the optimal CRT setting among the tested candidate CRT settings based on the output signal from the hemodynamic sensor. In the above presented example in Table 1 this corresponds to an AVD of 100 ms that maximizes the LV dP/dtₘₐₓ. This candidate CRT setting is then determined to be the optimal CRT setting for this CRT settings search period. In an alternative embodiment, the settings optimizer 131 calculates how the hemodynamic status changes over the range of tested candidate CRT settings. An optimal CRT setting is then determined to be the CRT setting that maximizes or minimizes (depending on which particular hemodynamic status parameter used) the sensor output.

The CRT settings search is then performed once more at a CRT settings search period, such as the consecutive week in order to identify the optimal CRT setting for that CRT settings search period.

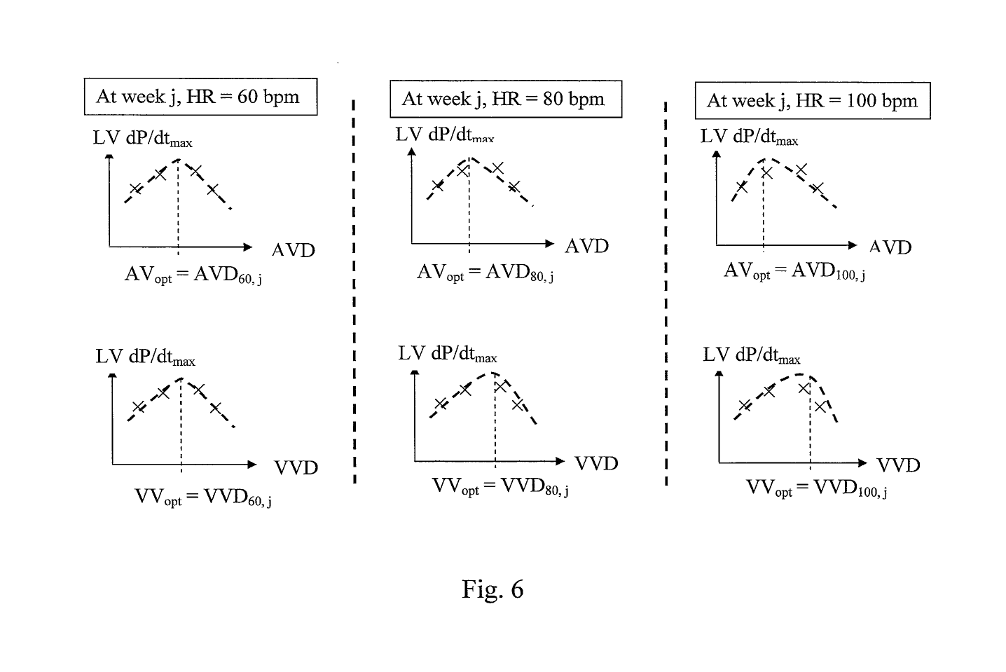

FIG. 6 illustrates this concept in the two left-most figures. Thus, at a given CRT setting search period at week j during the optimization time period five different candidate AVDs and candidate VVDs have been tested. The hemodynamic status is determined for each AVD and VVD illustrated by LV dP/dtₘₐₓ in the figure. Optimal AVD and VVD are determined for this week j by the settings optimizer 131 to maximize LV dP/dtₘₐₓ. The procedure is then once more conducted for a new week j+1 and so on until the end of the optimization time period.

Generally, the optimal CRT setting of the programmable CRT parameter is dependent on the heart rate of the subject's heart. Thus, for a given heart rate one CRT setting is optimal but for another heart rate another CRT setting could be more preferred. In such a case, the respective optimal CRT settings determined by the settings optimizer 131 at the different CRT settings search periods during the optimization time period are preferably determined for a same heart rate or at least a same heart rate range. Thus, it is not necessary that the heart rate is exactly the same at each CRT settings search period. Instead it is sufficient if the heart rate is at least within a defined heart rate range. The size of this heart rate range can be preconfigured in the IMD 100 or determined by the physician based on the particular subject. Generally, such a heart rate range could be defined as HR±10 bpm, HR±5 bpm or HR±2.5 bpm, where HR stands for the middle heart rate in bpm (beats per minute) of the heart rate range.

At the end of the optimization time period the IMD 100 therefore has determined and has access to multiple optimal CRT settings of the programmable CRT parameter determined for the defined heart rate range and for the respective CRT settings search periods. A settings predictor 132 is, in this embodiment, implemented in the IMD 100 and processes these multiple optimal CRT settings determined by the settings optimizer 131. In more detail, the settings predictor 132 determines at least one future optimal CRT setting based on the multiple optimal CRT settings. This means that the settings predictor 132 is capable of defining, based on the multiple optimal CRT settings, a trend in the CRT settings over time and is therefore able to predict or extrapolate how the optimal CRT settings will change in the future.

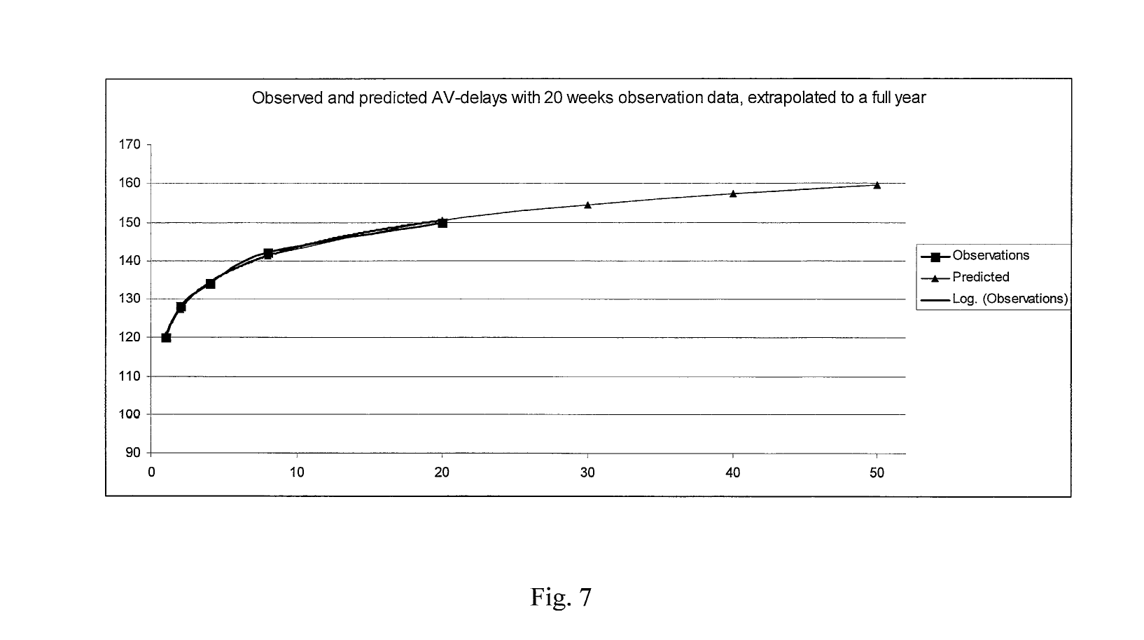

FIG. 7 schematically illustrates this concept with regard to observed and determined optimal AVD for 20 weeks, see full squares and thick line. Based on the determined optimal AVDs the settings predictor 132 is able to predict how the future optimal CRT will change over time, see full triangles and thin line.

The at least one future optimal CRT setting predicted by the settings predictor 132 is stored in a memory 170 of the IMD 100, where it is available to the controller 130. Following the end of the optimization time period the controller 130 can thereby select optimal CRT setting among the at least one future optimal CRT setting in the memory 170. Hence, no search for optimal CRT setting based on testing various CRT settings and using sensor feedback is therefore needed. This means that embodiments allow determining CRT settings for the IMD 100 even when the hemodynamic sensor no longer is operational or reliable. Furthermore, the embodiments save battery power for the IMD 100 since no CRT settings search procedure needs to be regularly initiated after the end of the optimization time period. In clear contrast, the only process needed is to fetch the optimal CRT setting from the memory 170. The controller 130 can therefore generate, following the end of the optimization time period, control signals to control the atrial and/or pulse generator 140, 150 to generate pacing pulses according to a CRT setting among the at least one future optimal CRT setting.

As is seen in FIG. 7, the CRT parameter could change over time to reach an asymptotic parameter value, i.e. slightly over 160 ms for the AVD example illustrated in the figure. In an embodiment, the settings predictor 132 is configured to predict a single future optimal CRT setting for the defined heart rate range and based on the determined optimal CRT settings. The settings predictor 132 could then advantageously determine the future optimal CRT setting to be the asymptotic CRT setting for the programmable CRT parameter. In such a case, it could be possible that the IMD 100 and the controller 130 will utilize a CRT setting that is not the most optimal one during a transition period following the end of the optimization time period. However, as time progresses the currently most optimal CRT setting will become ever closer to the value of the asymptotic CRT setting. Thus, for one or a few weeks the asymptotic CRT setting might not be the most optimal CRT setting but still be very close to optimal CRT setting.

In an alternative approach, the settings predictor 132 instead determines multiple future optimal CRT settings based on the optimal CRT settings and for the defined heart rate range. This is schematically illustrated in FIG. 7 showing three such future optimal CRT settings following the end of the optimization time period. In such a case, each future optimal CRT setting is applicable by the controller 130 at a respective time period following the end of the optimization time period. For instance and with reference to FIG. 7, the first future optimal CRT setting (AVD≈154 ms) could be applicable the first 15 weeks following the end of the optimization time period, the second future optimal CRT setting (AVD≈157 ms) is used from week 15 to week 25 from the end of the optimization time period. The asymptotic CRT setting (AVD≈160 ms) could then be used after the end of week 25 from the end of the optimization time period.

Thus, each of the multiple future optimal CRT settings predicted by the settings predictor 132 is preferably associated with a respective time period during which the future optimal CRT setting can be used by the controller 130. The controller 130 therefore selects which of the multiple future optimal CRT settings to be used based on the length of the time period lapsed since the end of the optimization time period.

The settings predictor 132 can be configured to predict multiple, discrete future optimal CRT settings, such as the three full triangles in FIG. 7 following the end of the optimization time period. In an alternative approach, the settings predictor 132 defines, based on the multiple optimal CRT settings determined by the settings optimizer 131, a mathematical function that outputs a future optimal CRT setting based on a current length of a time period lapsed since the end of the optimization time period or the start of the optimization time period. The settings predictor 132 preferably fits a mathematical function that minimizes the difference between the function outputs and the determined optimal CRT settings in manner that is well known in the art of function fitting. FIG. 7 illustrates this concept with the thin line corresponding to the mathematical function 10.01 ln(t)+120.48, where t represents the time in weeks. With this embodiment, the controller 130 can calculate the future optimal CRT setting to use at each particular time instance by simply inputting the current time lapsed since the end of the optimization time period or the start of the optimization time period.

As was briefly mentioned above, the optimal CRT setting of the at least one programmable CRT parameter is typically dependent on the heart rate of the subject. In a particular embodiment, the IMD 100 determines future optimal CRT settings that are applicable for different heart rate ranges to thereby have multiple sets of optimal CRT settings to select among based on the subject's current heart rate.

The controller 130 is then configured to control the atrial and/or ventricular pulse generator 140, 150 to generate pacing pulses according to multiple different candidate CRT settings of the programmable CRT parameter for multiple different heart rate ranges at the multiple CRT settings search periods. Thus, at each time interval when the controller 130 is to conduct a CRT settings search multiple candidate CRT settings are tested for each of the multiple different heart rate ranges. For instance, a first such heart rate range could be 60±10 bpm or 70 bpm, a second heart rate range is 80±10 bpm and a third heart rate range could be 100±10 bpm or ≧90 bpm. The number of heart rate ranges and the respective sizes of the heart rate ranges can be preconfigured in the IMD 100 or programmed by the physician depending on the particular CRT parameter.

The settings optimizer 131 is configured, in this embodiment, to determine a respective optimal CRT setting for each heart rate range and for each CRT settings search period based on the multiple candidate CRT settings tested and the output signals from the hemodynamic sensor. FIG. 6 illustrates this concept for one such time interval (week j). The figure illustrates determining optimal AVDs and VVDs for three different heart rate ranges resulting in three optimal CRT settings per CRT parameter for this particular CRT settings search period, i.e. week j.

In this embodiment the settings predictor 132 predicts at least one future optimal CRT setting for each heart rate range based on the respective optimal CRT settings for that heart rate range determined by the settings optimizer 131. The prediction of at least one future optimal CRT setting can be conducted as previously described herein but in parallel or in series for the respective heart rate ranges. The result could then be a single (asymptotic) CRT setting, multiple, discrete future optimal CRT settings, or a mathematical function outputting a future optimal CRT setting for each of the heart rate ranges. The respective at least one future optimal CRT settings are stored in the memory 170.

The controller 130 can then generate control signal following the end of the optimization time period to control the atrial and/or ventricular pulse generator 140, 150 to generate pacing pulses according to a CRT setting selected from the memory 170 by the controller 130 based on a current heart rate of the subject's heart. Thus, the controller 130 receives input signals from a unit, such as an intracardiac electrogram (IEGM) unit 160 of the IMD 110, where the input signals are representative of the current heart rate. This heart rate is used to identify the set of at least one future optimal CRT settings to use, i.e. the heart rate is within the heart rate range for which the set is applicable. The controller 130 then retrieves the future optimal CRT setting therefrom if the set only comprises a single future optimal CRT setting or selects or calculates the future optimal CRT setting from the set based on the current length of the time period lapsed since the end of the optimization time period if the set comprises multiple future optimal CRT settings or defines a mathematical function.

If the subject will not, at each CRT settings search period at which a CRT settings search is to be conducted, reach the respective heart rate ranges various measures can be taken. In a first embodiment, no optimal CRT setting is simply determined for the given heart rate range and given CRT settings search period. This is generally no problem if optimal CRT setting can be determined for the given heart rate range at the other CRT settings search periods during the optimization time period. In an alternative approach, the CRT settings searches are conducted in connection with visits at the physician. The subject can then be asked to exercise slightly, for instance on a bike or treadmill, in order to reach higher heart rate ranges than the resting heart rate. Such a procedure therefore forces the subject's heart rate into higher regions, for which optimal CRT settings can be determined. In yet an alternative approach, the IMD 100 and its controller 130 can temporary program a higher base heart rate before carrying to the CRT settings searches to reach the different heart rate ranges.

It is also possible to calculate future optimal CRT settings for certain heart rate ranges based on the future optimal CRT settings predicted for at least two different heart rate ranges. In such a case, the settings predictor 132 predict, for at least one heart rate range, at least one future optimal CRT setting based on the future optimal CRT settings predicted by the settings predictor 132 for at least two different heart rate ranges. For instance, if the settings predictor 132 has predicted future optimal CRT settings for the heart rate range 60±10 bpm and the heart rate range 100±10 bpm it could calculate future optimal CRT settings for the heart rate range 80±10 bpm as an average of the future optimal CRT settings for these two heart rate ranges. Depending on which heart rate range to calculate future optimal CRT settings for and which heart rate ranges that have available predicted future optimal CRT settings, the settings predictor 132 can calculate the at least one future optimal CRT setting by interpolating or extrapolating from the available heart rate ranges. It is of course possible to use future optimal CRT settings from more than two heart rate ranges when calculating the at least one future optimal CRT setting for another heart rate range.

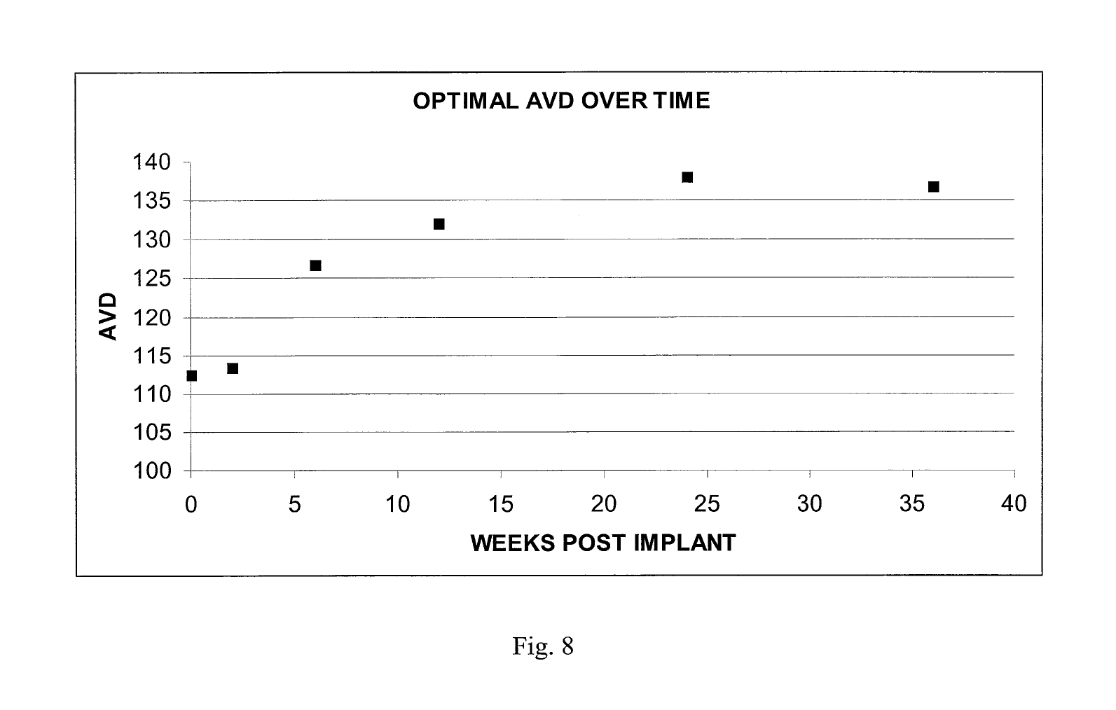

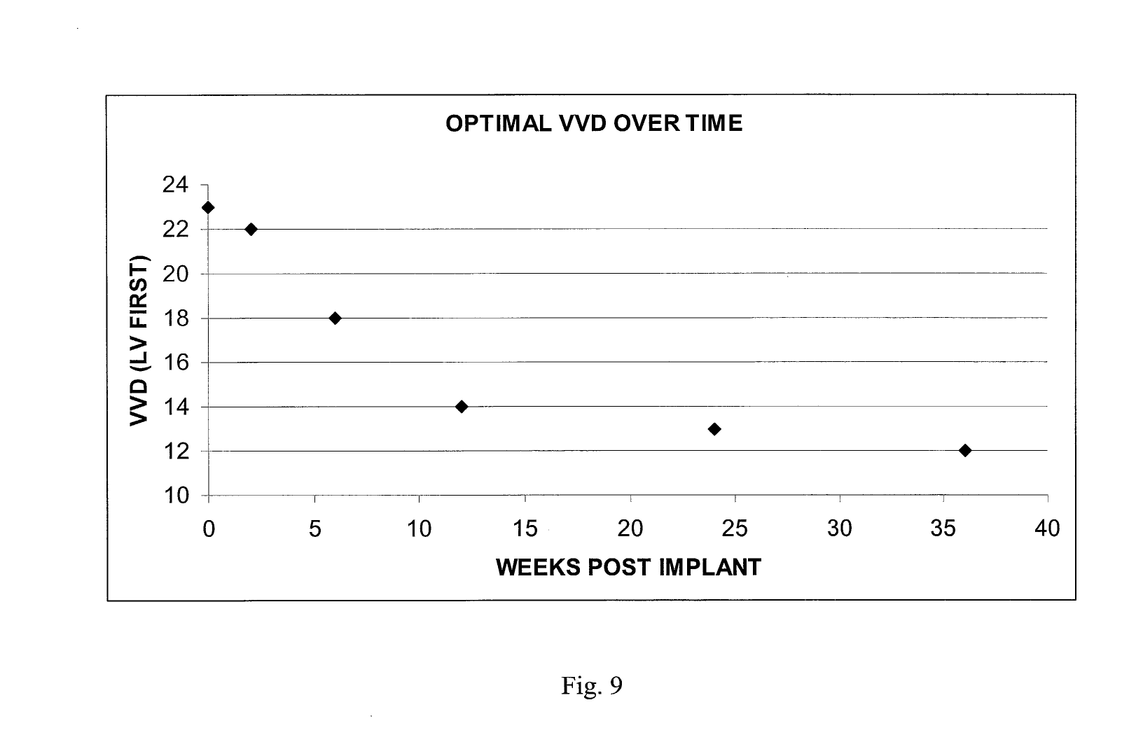

FIGS. 8 and 9 illustrate the change in optimal AVD and VVD over time following implantation of an IMD illustrated as an average in a group of 40 CRT patients (data extracted from Pacing and Clinical Electrophysiology, vol. 28, pages S24-S26, 2005). It is seen from these figures that the optimal AVD and VVD drift or change over time, probably due to remodulations in the patients following implantation. At about 2-3 months from the implantation date the optimal AVD and VVD stabilize and do not change that much further over time. Hence, there is clinical data available which supports that the embodiments can be successful in predicting future optimal CRT settings based on previously determined optimal CRT settings since there is a predictable function in how the optimal CRT settings change over time.

The determination or selection of optimal CRT setting is conducted, according to the embodiments, based on the output signal from a hemodynamic sensor and is therefore dependent on the resulting hemodynamic status of the subject. Various types of hemodynamic sensors can be used according to the embodiments. An example of hemodynamic sensor is a pressure sensor configured to generate an output signal representative of left ventricular

$$( LV ) {( \frac{d P}{d t} )}_{ma x} .$$

Various implantable, chronic pressure sensors are available on the market and can be used according to the embodiments. A problem with most such implantable pressure sensors is that they become inoperable and unpredictable after some time in the subject body. However, as the embodiments only need sensor readings during the optimization time period immediately following implantation and extending up to, preferably at least 2-3 months it is possible to use implantable pressure sensors that can be used for at least this time period. A non-limiting example of such a pressure sensor that can be used includes Radi PressureWire®. In such a case, the pressure sensor is preferably present in the right ventricle (RV), either as a separate pressure wire or catheter or having a pressure sensor integrated onto the right ventricular lead.

Another example of sensor technique that can be used is a cardomechanical electric sensor (CMES) configured to generate an output signal representative of

$${LV ( \frac{d P}{d t} )}_{ma x} .$$

Such a CMES sensor that can be used according to the embodiments is disclosed in US 2009/0312814 and US 2009/0253993. The CMES is advantageously positioned in a coronary vein to produce a signal that can be processed to get an output signal representative of

$${LV ( \frac{d P}{d t} )}_{ma x} .$$

Another type of sensor that can produce an output signal representative of

$${LV ( \frac{d P}{d t} )}_{ma x}$$

is an accelerometer. In such a case, the accelerometer is preferably configured and implanted to register peak endocardial acceleration. The accelerometer output signal is in essence a high-pass filtered pressure signal that is representative of

$${LV ( \frac{d P}{d t} )}_{ma x} .$$

In the art, such an accelerometer are denoted SonR sensors and have been suggested for usage in connection with hemodynamic monitoring of CRT patients by being placed either in the right atrium or the right ventricle, see Pacing and Clinical Electrophysiol, vol 32, pages S240-S246, 2009.

Another sensor type that can be used to monitor the hemodynamic status of the subject is a flow sensor preferably configured to be arranged in connection with the descending aorta of the subject. The output signal from the flow sensor will then be representative of the

$${LV ( \frac{d P}{d t} )}_{ma x}$$

of the subject since the flow sensor can register the change in blood flow out of the aorta valve and this change in blood flow is proportional to

$${LV ( \frac{d P}{d t} )}_{max} .$$

Various types of flow sensors can be used including optical flow sensors, mechanical flow sensors, etc. The flow correlates well with the early phases of systole

$${LV ( \frac{d P}{d t} )}_{max} .$$

The flow sensor can also be used to generate an output signal that is representative of the stroke volume, which is another hemodynamic status parameter that can be used according to the embodiments.

Also an acoustic sensor, such as microphone, capturing heart sound can be used to generate an output signal representative of

$${LV ( \frac{d P}{d t} )}_{max} .$$

Heart sound measurements can be made to correlate to

$${LV ( \frac{d P}{d t} )}_{max}$$

by calculating the power of the S1 heart sound in the sensor output signal. The acoustic sensor is advantageously position at the can or case of the IMD or could be placed on the right ventricular lead.

Finally, an impedance sensor can be used, i.e. calculating the impedance based on an electric signal applied over two electrodes and the resulting electric signal senses over two electrodes, as a representation of

$${LV ( \frac{d P}{d t} )}_{max} .$$

The impedance sensor then preferably uses an impedance vector that captures the blood volume change in the left ventricle or the right ventricle. Non-limiting examples of suitable impedance vectors include 1) i: RV ring-LV ring, u: RV tip-LV tip; 2) i: RV ring-LV ring, u: RV tip-LV ring; 3) i: RV coil-LV ring, u: RV coil-LV tip. It is then possible to determine the change in blood volume in the ventricle over time, i.e.

$${( - \frac{d V}{d t} )}_{max}$$

which can be used as a surrogate for

$${ILV ( \frac{d P}{d t} )}_{max} .$$

Another impedance vector, SVC coil—can/case is suitable for determining an impedance signal that is representative of the stroke volume.

The determination or selection of optimal CRT setting is conducted, according to the embodiments, based on the output signal from a hemodynamic sensor and is therefore dependent on the resulting hemodynamic status of the subject. Various types of hemodynamic sensors can be used according to the embodiments. Thus, the embodiments are not limited to left ventricular

$${( \frac{d P}{d t} )}_{max}$$

as status parameter. An example of another such hemodynamic parameter is the stroke volume, i.e. the volume of blood pumped from a ventricle of the heart with each beat. Actually, any status parameter representative of the hemodynamic status of a subject and that can be registered or monitored by an implantable hemodynamic sensor can be used by the embodiments.

In FIG. 2, the IMD 100 has been exemplified by a processing unit 133 that is configured to determine left ventricular

$${( \frac{d P}{d t} )}_{max}$$

based on the output signals from the hemodynamic sensor connectable to the IMD 100 through the terminal 117.

An optional electronic configuration switch 120 includes a plurality of switches for connecting the desired terminals 111-117 to the appropriate I/O circuits, thereby providing complete electrode programmability. Accordingly, the electronic configuration switch 120, in response to a control signal from the controller 130, determines the polarity of the stimulating pulses (e.g., unipolar, bipolar, combipolar, etc.) by selectively closing the appropriate combination of switches (not shown) as is known in the art.

An optional atrial sensing circuit or detector 145 and a ventricular sensing circuit or detector 155 are also selectively coupled to the atrial lead(s) and the ventricular lead(s) through the switch 120 for detecting the presence of cardiac activity in the heart chambers. Accordingly, the atrial and ventricular sensing circuits 145, 155 may include dedicated sense amplifiers, multiplexed amplifiers, or shared amplifiers. The switch 120 determines the “sensing polarity” of the cardiac signal by selectively closing the appropriate switches, as is also known in the art. In this way, the clinician may program the sensing polarity independent of the stimulation polarity. The sensing circuits are optionally capable of obtaining information indicative of tissue capture.

Each sensing circuit 145, 155 preferably employs one or more low power, precision amplifiers with programmable gain and/or automatic gain control, band-pass filtering, and a threshold detection circuit, as known in the art, to selectively sense the cardiac signal of interest.

The outputs of the atrial and ventricular sensing circuits 145, 155 are connected to the controller 130, which, in turn, is able to trigger or inhibit the atrial and ventricular pulse generators 140, 150, respectively, in a demand fashion in response to the absence or presence of cardiac activity in the appropriate chambers of the heart.

Furthermore, the controller 130 is also typically capable of analyzing information output from the sensing circuits 145, 150 and/or the IEGM unit 160 to determine or detect whether and to what degree tissue capture has occurred and to program a pulse, or pulse sequence, in response to such determinations. The sensing circuits 145, 155, in turn, receive control signals over signal lines from the controller 130 for purposes of controlling the gain, threshold, polarization charge removal circuitry, and the timing of any blocking circuitry coupled to the inputs of the sensing circuits 145, 155 as is known in the art.

Cardiac signals are applied to inputs of the IEGM unit 160 connected to the lead and sensor connector 110. The IEGM unit 160 is preferably in the form of an analog-to-digital (ND) data acquisition unit configured to acquire IEGM signals, convert the raw analog data into a digital signal, and store the digital signals for later processing and/or transmission to the programmer by a transmitter 190. The IEGM unit 160 is coupled to the atrial lead and/or the ventricular lead through the switch 120 to sample cardiac signals across any pair of desired electrodes.

Advantageously, the operating parameters of the IMD 100 may be non-invasively programmed into the memory 170 through the receiver or transceiver 190 in communication via a communication link with the previously described communication unit of the programmer. The controller 130 activates the transceiver 190 with a control signal. The transceiver 190 can alternatively be implemented as a dedicated receiver and a dedicated transmitter connected to separate antennas or a common antenna, preferably a radio frequency (RF) antenna 195.

The IMD 100 additionally includes a battery 180 that provides operating power to all of the circuits shown in FIG. 2.

In the figure the settings optimizer 131, the settings predictor 132 and the processing unit 133 have been exemplified as being run by the controller 130.

These units can then be implemented as a computer program product stored on the memory 170 and loaded and run on a general purpose or specially adapted computer, processor or microprocessor, represented by the controller 130 in the figure. The software includes computer program code elements or software code portions effectuating the operation of the settings optimizer 131, the settings predictor 132 and the processing unit 133. The program may be stored in whole or part, on or in one or more suitable computer readable media or data storage means that can be provided in an IMD 100.

In an alternative embodiment, the settings optimizer 131, the settings predictor 132 and the processing unit 133 are implemented as hardware units either forming part of the controller 130 or provided elsewhere in the IMD 100.

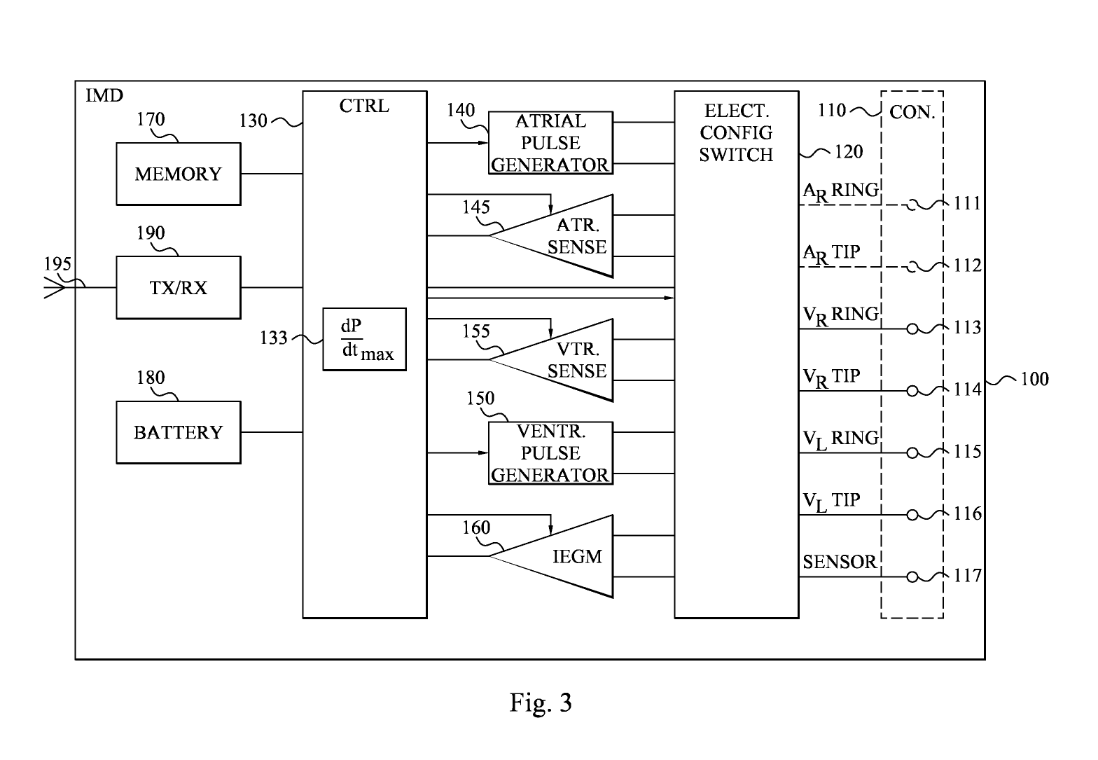

In an alternative embodiment, the system for determining CRT settings for an IMD comprises both the IMD and the external data processing unit as illustrated in FIG. 1. FIG. 3 schematically illustrates an embodiment of the IMD 100 in the system. Compared to the embodiment illustrated in FIG. 2, the settings optimizer and the settings predictor are absent in the IMD 100. In clear contrast, in this embodiment information of the tested candidate CRT settings and the processing results from the processing unit 133 in terms of hemodynamic status of the subject are either stored in the memory 170 for later transmission to the data processing unit or are directly uploaded to the data processing unit by the transmitter 190 and transmit antenna 195. The processing of this information in terms of determining respective optimal CRT setting for the defined heart rate range(s) and for each CRT settings search period and the prediction of at least one future optimal CRT setting are then conducted in the data processing unit. The result of the processing, i.e. the at least one future optimal CRT setting, is then downloaded and programmed into the IMD 100 by transmitting information of the at least one future optimal CRT setting and preferably information of the heart rate range(s) it/they is/are applicable for and preferably time information defining during which time intervals following the end of the optimization time period it/they is/are applicable. This information is then entered in the memory 170 and can be used by the controller 130 as previously described for the purpose of generating control signals to control the atrial and/or ventricular pulse generator 140, 150 to generate pacing pulses according to a future optimal CRT setting.

The operation of the remaining units in the IMD 100 of FIG. 3 is basically the same as for the IMD 100 of FIG. 2.

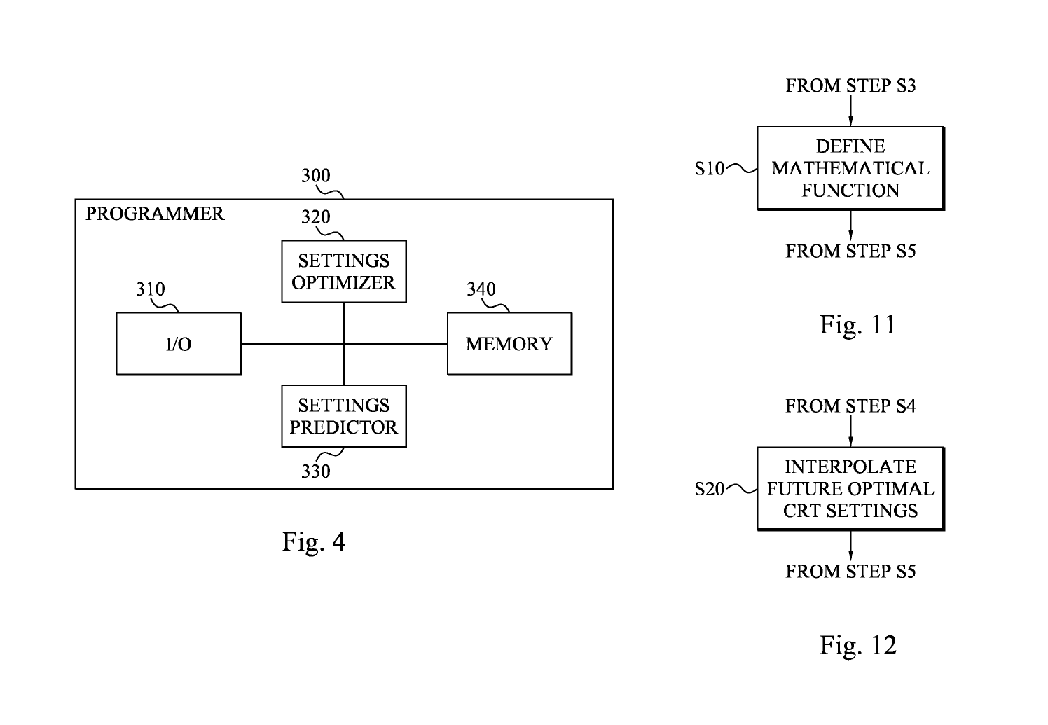

FIG. 4 is a schematic block diagram of the non-implantable data processing device 300, exemplified as a programmer in the figure. The data processing device 300 comprises a unit 310 capable of conducting communication with the IMD. This unit 310 can either be a transceiver or a transmitter and receiver pair with connected antenna(s). In an alternative approach, the data processing device 300 is configured to be connected to a communication device (see FIG. 1) that performs the wireless communication with the IMD on behalf of the data processing device 300. In such a case, the unit 310 can be a general input and output (I/O) unit 310 that is in wired or wireless connection with the communication device. The unit 310 therefore receives information of the output signals from the hemodynamic sensor, i.e. information of the hemodynamic status of the subject. In addition, the unit 310 preferably also receives information of which candidate CRT settings that have been tested and which are associated with the particular hemodynamic status information. In an alternative approach, the IMD is preconfigured to test a defined set of candidate CRT settings. In such a case, this information of the defined set of candidate CRT settings can be stored in a memory 340 of the data processing device 300 and therefore does not need to be received from the IMD.

The data processing device 300 also comprises, in this embodiment, the settings optimizer 320 and the settings predictor 330. The operation of the settings optimizer 320 and the settings predictor 330 is basically the same as when these units would have been implemented in the IMD as illustrated in FIG. 2. The discussion of these units in the foregoing therefore also applies herein when being arranged in the data processing device 300.

The output of the settings predictor 330, i.e. information of the at least one future optimal CRT setting, is either directly transmitted to the IMD through the unit 310 or is first stored in the memory 340 and then later on downloaded to the IMD.

In this embodiment the data processing operations are conducted in the data processing device 300 and not in the IMD, which thereby saves processing capacity for the controller and power of the IMD battery. However, the embodiment also requires more signaling between the IMD and the data processing device.

In alternative embodiments, the settings optimizer could be arranged in the IMD and the settings predictor in the data processing device or the settings predictor is arranged in the IMD and the settings optimizer in the data processing device.

The units 310 to 330 of the data processing device 300 may be implemented or provided as hardware or a combination of hardware and software. In the case of a software-based implementation, a computer program product implementing the units 310 to 330 or a part thereof comprises software or a computer program run on a general purpose or specially adapted computer, processor or microprocessor. The software includes computer program code elements or software code portions illustrated in FIG. 4. The program may be stored in whole or part, on or in one or more suitable computer readable media or data storage means such as magnetic disks, CD-ROMs, DVD disks, USB memories, hard discs, magneto-optical memory, in RAM or volatile memory, in ROM or flash memory, as firmware, or on a data server.

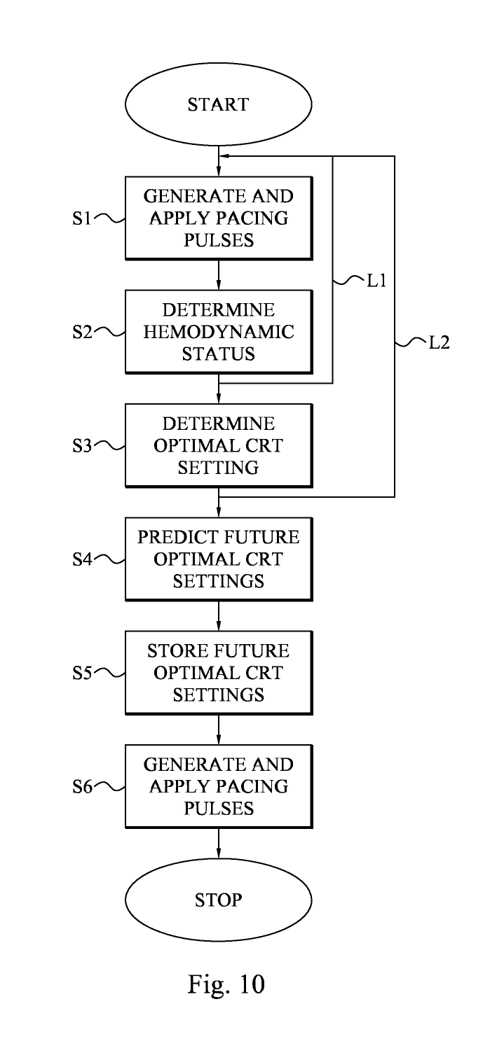

FIG. 10 is a flow diagram illustrating a method of determining CRT settings for an IMD. The method starts in step S1 where the IMD generates and applies pacing pulses to a first heart chamber and a second heart chamber of a subject's heart according to a candidate CRT setting of a programmable CRT parameter of the IMD. A hemodynamic sensor generates, in step S2, a signal representative of the hemodynamic status of the subject caused by pacing according to the candidate CRT setting.

Steps S1 and S2 are repeated for multiple different candidate CRT settings at a CRT settings search period, which is schematically illustrated by the line L1.

A next step S3 determines the optimal CRT setting based on the multiple different candidate CRT settings and also based on the output signals form the hemodynamic sensor. Thus, step S3 preferably selects the candidate CRT setting that leads to most optimal or best the hemodynamic status as determined based on the sensor output signals. This optimal CRT setting is preferably determined and applicable for a defined heart rate range.

The loop of steps S1 to S3 is then repeated for another CRT settings search period during an optimization time period, which is schematically illustrated by the line L2. This means that an optimal CRT setting is preferably determined for the defined heart rate range for CRT settings search period, such as week, during the optimization time period.

The following step S4 predicts at least one future optimal CRT setting for the defined heart rate range based on the determined optimal CRT settings. This at least one future optimal CRT setting is stored in a memory of the IMD in step S5. The IMD then generates and applies pacing pulses to the first and second heart chambers following the end of the optimization time period according to a CRT setting of the at least one future optimal CRT settings in step S6.

In a particular embodiment of step S4, multiple future optimal CRT settings are predicted for the defined heart rate range, where each such future optimal CRT setting is applicable during a respective time interval following the end of the optimization time period. In such a case, the IMD generates and applies pacing pulses according to a CRT setting selected among the multiple future optimal CRT settings based on a current length of the time period lapsed since the end of the optimization time period.

FIG. 11 illustrates a particular embodiment of step S4. In this case, the multiple optimal CRT settings determined in step S3 for the defined heart rate range and the multiple CRT settings search periods are used in step S10 to define a mathematical function that defines the optimal CRT setting as a function of time. Step S10 preferably involves fitting a mathematical function to the multiple optimal CRT settings, such as a mathematical function defined as f(t)=a ln(t)+b. The fitting procedure then defines the parameters a, b in order to minimize the error between the function f(t) and the multiple optimal CRT settings. The parameters of the function are then stored in step S5 and the IMD generates and applies pacing pulses according to a CRT setting obtained from the mathematical function based on a current length of the time period lapsed since the end of the optimization time period or at the start of the optimization time period.

According to a particular embodiment steps S1 and S2 are repeated for multiple different heart rate ranges to thereby test candidate CRT settings applicable to different heart rate ranges. This means that during each CRT settings search period a set of multiple candidate CRT settings is tested for multiple different heart rate ranges. Steps S3 then determines a respective optimal CRT setting for each heart rate range. At the end of the optimization period a set of multiple optimal CRT settings are then available for the respective heart rate ranges, where the respective optimal CRT settings have been determined at different CRT settings search periods. Step S4 therefore involves predicting at least one future optimal CRT settings for each of the multiple heart rate ranges based on the multiple optimal CRT settings determined for that heart rate range. These multiple future optimal CRT settings are stored in step S5 in the IMD memory. The IMD then generates and applies pacing pulses to the heart according to a CRT setting selected among the stored future optimal CRT settings based on a current heart rate and preferably also based on a current length of the time period lapsed since the end of the optimization time period.

FIG. 12 is a flow diagram of an additional, optional step of the method in FIG. 10. The method continues from step S4 in FIG. 10. A next step S20 predicts, for a defined heart rate range, at least one future optimal CRT setting based on the future optimal CRT settings predicted for at least two different heart rate ranges. This prediction in step S20 can be conducted to extrapolate or interpolate the at least one future optimal CRT setting dependent on whether the defined heart rate range is larger/smaller than the two different heart rate ranges or in between the two different heart rate ranges. The method then continues to step S5, where the predicted at least one future optimal CRT setting is stored in the IMD memory.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1-15. (canceled)

16. A system for determining cardiac resynchronization therapy, CRT, settings for an implantable medical device, the system comprising:
a first cardiac lead implantable in or in connection with a first chamber of a heart of a subject and having at least one pacing and sensing electrode,
a second cardiac lead implantable in or in connection with a second chamber of said heart and having at least one pacing and sensing electrode,
a hemodynamic sensor configured to generate output signals representative of a hemodynamic status of said subject;
at least one pacing pulse generator coupled to said first and second cardiac leads and sensor and configured to generate pacing pulses; and
a controller coupled to said at least one pacing pulse generator and configured to generate control signals to control said at least one pacing pulse generator to generate pacing pulses according to multiple different candidate CRT settings of a programmable CRT parameter;
a settings optimizer configured to determine, for a defined heart rate range of said heart a respective optimal CRT setting based on said multiple different candidate CRT settings and based on said output signals from said hemodynamic sensor during an optimization time period; and
a settings predictor configured to predict, for said defined heart rate range, at least one future optimal CRT setting based on said respective optimal CRT settings determined by said settings optimizer during an optimization time period, wherein said implantable medical device further comprises a memory for storing said at least one future optimal CRT setting and said controller is configured to generate, following an end of the optimization time period, control signals to control said at least one pacing pulse generator to generate pacing pulses according to a CRT setting of said at least one future optimal CRT setting.

17. The system according to claim 16, wherein
said settings predictor is configured to predict, for said defined heart rate range, multiple future optimal CRT settings based on said respective optimal CRT settings determined by said settings optimizer, wherein each future optimal CRT setting of said multiple future optimal CRT settings is applicable by said controller at a respective time period following said end of said optimization time period; and
said controller is configured to generate, following said end of said optimization time period, control signals to control said at least one pacing pulse to generate pacing pulses according to a CRT setting settings selected by said controller (130) among said multiple future optimal CRT based on a current length of a time period lapsed since said end of said optimization time period.

18. The system according to claim 16, wherein said settings predictor is configured to define a mathematical function outputting a future optimal CRT setting based on a current length of a time period lapsed since said end of said optimization time period by fitting said mathematical function to said multiple optimal CRT settings determined by said settings optimizer.

19. The system according to claim 16, wherein
said controller is configured to generate control signals to control said at least one pacing pulse generator to generate, at said multiple CRT settings search periods during said optimization time period and for multiple different heart rate ranges of said heart, pacing pulses according to multiple different candidate CRT settings of said programmable CRT parameter;
said settings optimizer is configured to determine, for each heart rate range of said multiple different heart rate ranges and for each CRT settings search period of said multiple CRT settings search periods, a respective optimal CRT setting based on said multiple different candidate CRT settings and based on said output signal from said hemodynamic sensor;
said settings predictor is configured to predict, for each heart rate range of said multiple different heart rate ranges, at least one future optimal CRT setting based on said respective optimal CRT settings for said heart rate range determined by said settings optimizer;
said memory is configured to store, for each heart rate range of said multiple different heart rate ranges, said at least one future optimal CRT setting; and
said controller is configured to generate, following said end of said optimization time period, control signals to control said at least one pacing pulse generator to generate pacing pulses according to a CRT setting selected by said controller among said future optimal CRT settings based on a current heart rate of said heart.

20. The system according to claim 19, wherein
said settings predictor is configured to predict, for at least one defined heart rate range, at least one future optimal CRT setting based on future optimal CRT settings predicted by said setting predictor for at least two different heart rate ranges of said multiple different heart rate ranges; and
said memory is configured to store, for said at least one defined heart rate range, said at least one future optimal CRT setting predicted by said settings predictor.

21. The system of claim 16, wherein said hemodynamic sensor is selected from the group consisting of a pressure sensor configured to generate an output signal representative of left ventricular
$${( \frac{d P}{d t} )}_{max}$$
of said subject (5), a cardiomechanical electric sensor, CMES, configured to generate an output signal representative of left ventricular
$${( \frac{d P}{d t} )}_{max}$$
of said subject (5), an accelerometer configured to generate an output signal representative of left ventricular
$${( \frac{d P}{d t} )}_{max}$$
of said subject (5), a flow sensor configured to be arranged in connection with the descending aorta of said subject (5) and generate an output signal representative of left ventricular
$${( \frac{d P}{d t} )}_{max}$$
of said subject (5), a microphone configured to capture heart sound from said subject (5) and generate an output signal representative of left ventricular
$${( \frac{d P}{d t} )}_{max}$$
of said subject (5) and an impedance sensor configured to generate an output signal representative of left ventricular
$${( - \frac{d V}{d t} )}_{max}$$
of said subject.

22. The system according to claim 16, wherein said implantable medical device comprises said settings optimizer and said settings predictor.

23. The system according to claim 16, wherein said settings optimizer and said settings predictor are implemented in a non-implantable data processing device and said implantable medical device comprises:
a transmitter configured to transmit, to said non-implantable data processing device, information of said output signals from said hemodynamic sensor; and
a receiver configured to receive, from said non-implantable data processing device, information of said at least one future optimal CRT setting.

24. The system according to claim 16, wherein said first heart chamber is a right ventricle of said heart and said second heart chamber is a left ventricle of said heart and said programmable CRT parameter is an interventricular delay.

25. The system according to claim 16, wherein said first heart chamber is a ventricle of said heart and said second heart chamber is an atrium of said heart and said programmable CRT parameter is an atrioventricular delay.

26. A method for determining cardiac resynchronization therapy, CRT, settings for an implantable medical device comprising:
a) said implantable medical device generating and applying pacing pulses to a first heart chamber of a heart of a subject and a second heart chamber of said heart according to a CRT setting of a programmable CRT parameter of said implantable medical device;
b) a hemodynamic sensor generating output signals representative of a hemodynamic status of said subject;
c) repeating steps a) and b) for multiple different candidate CRT settings of said programmable CRT parameter at a CRT settings search period during an optimization time period;
d) determining, for a defined heart rate range of said heart (10), an optimal CRT setting based on said multiple different candidate CRT settings and based on said output signals from said hemodynamic sensor;
e) repeating steps a) to d) for multiple CRT settings search periods during said optimization time period;
f) predicting, for said defined heart rate range, at least one future optimal CRT setting based on said determined optimal CRT settings;
g) storing said at least one future optimal CRT setting in a memory of said implantable medical device; and
h) said implantable medical device generating and applying, following an end of said optimization time period, pacing pulses to said first heart chamber said second heart chamber according to a CRT setting of said at least one future optimal CRT settings.

27. The method according to claim 26, wherein
step f) comprises predicting, for said defined heart rate range, multiple future optimal CRT settings based on said determined optimal CRT settings, wherein each future optimal CRT setting of said multiple future optimal CRT settings is applicable at a respective time period following said end of said optimization time period, and
step h) comprises said implantable medical device generating and applying, following said end of said optimization time period, pacing pulses to said first heart chamber said second heart chamber according to a CRT setting selected among said multiple future optimal CRT setting based on a current length of a time period lapsed since said end of said optimization time period.

28. The method according to claim 26, further comprising defining a mathematical function outputting a future optimal CRT setting based on a current length of a time period lapsed since said end of said optimization time period by fitting said mathematical function to said multiple optimal CRT settings, wherein step h) comprises said implantable medical device generating and applying, following said end of said optimization time period, pacing pulses to said first heart chamber said second heart chamber according to a CRT setting obtained from said mathematic function based on a current length of a time period lapsed since said end of said optimization time period.

29. The method according to claim 26, further comprising:
i) repeating steps a) and b) for multiple different heart rate ranges of said heart, wherein
step c) comprises repeating steps a), b) and i) for multiple different candidate CRT settings of said programmable CRT parameter at a CRT settings search period during an optimization time period;
step d) comprises determining, for each heart rate range of said multiple different heart rate ranges, an optimal CRT setting based on said multiple different candidate CRT settings and based on said output signal from said hemodynamic sensor (240);
step e) comprises repeating steps a), b), i), c) and d) for multiple CRT settings search periods during said optimization time period;
step f) comprises predicting, for each heart rate range of said multiple heart rate ranges, at least one future optimal CRT setting based on said determined optimal CRT settings;
step g) comprises storing, for each heart rate range of said multiple different heart rate ranges, said at least one future optimal CRT setting in said memory of said implantable medical device; and
step h) comprises said implantable medical device generating and applying, following said end of said optimization time period, pacing pulses to said first heart chamber said second heart chamber according to a CRT setting selected among said future optimal CRT settings based on a current heart rate of said heart.

30. The method according to claim 29, further comprising
predicting, for at least one defined heart rate range, at least one future optimal CRT setting based on future optimal CRT settings predicted for at least two different heart rate ranges of said multiple different heart rate ranges; and
storing, for said at least one defined heart rate range, said at least one future optimal CRT setting in said memory of said implantable medical device.
